(19) United States
(12) Patent Application Publication
(10) Pub. No.: US 2018/0347055 A1
(43) Pub. Date: Dec. 6, 2018

(54) ELECTROCHEMICAL SYNTHESIS OF DICARBAMATES

(71) Applicant: Covestro Deutschland AG, Leverkusen (DE)

(72) Inventors: Frank Richter, Leverkusen (DE); Heike Heckroth, Odenthal (DE); Vinh Trieu, Köln (DE); Marta Feroci, Rom (IT); Gianpiero Forte, Formia (IT); Achille Inesi, Rom (IT); Isabella Chiarotto, Rom (IT)

(21) Appl. No.: 15/527,901

(22) PCT Filed: Nov. 25, 2015
(86) PCT No.: PCT/EP2015/077695

§ 371 Date: May 18, 2017

(30) Foreign Application Priority Data
Nov. 28, 2014 (IT) RM2014A000694

(51) Int. Cl. C25B 3/04; C07C 269/04

(52) U.S. Cl. CPC C25B 3/04; C07C 2601/14; C07C 269/04

(57) ABSTRACT

The invention relates to an electrochemical process for preparing bis-O-alkyl-carbamates from primary amines with CO₂ as carbonyl source and at least one alkyl halide with at least three carbon atoms in the alkyl group as alkylating agent.

### CROSS-REFERENCE TO RELATED APPLICATIONS

This Application is a National Phase Application of PCT/EP2015/077695, filed Nov. 25, 2015, which claims priority to Italian Application No. RM2014A000694, filed Nov. 28, 2014 each of which are incorporated herein by reference.

### FIELD OF THE INVENTION

Several chemical routes for the production of O-alkyl carbamates as precursors for isocyanates are known, e.g. by reaction of primary amines with urea as the carbonyl source and an alcohol and evolution of ammonia. Employing an electrochemical route has the advantage that CO₂ as sustainable raw material can be used directly as the carbonyl source and no ammonia is produced as a side-product.

### BACKGROUND OF THE INVENTION

It is known that mono O-alkyl-carbamates can be synthesized electrochemically from mono amines with CO₂ as the carbonyl source and an alkyl halide as alkylating agent. The synthesis of bis-O-alkyl-carbamates from primary diamines is not described in the prior art.

Furthermore, the general disadvantage of the electrochemical routes of the state of the art is that ethyl chloride (Zeitschrift fuer Chemie 1988, 28, 372-373 and Pharmazie 1992, 47, 848-851) or ethyl iodide (Chem. Commun., 1996, 2575-2576; J. Org. Chem. 2007, 72, 200-203; J. Org. Chem. 1997, 62, 6754-6759; Electrochim. Acta 2011, 56, 5823-5827; Tetrahedron Lett. 2000, 41, 963-966; J. Org. Chem. 2003, 68, 1548-1551 and Appl. Organometal. Chem. 2007, 21, 941-944.), respectively, are used as alkylating agent. Both are impractical to use on a large scale for price, boiling point, and toxicological considerations.

The reactions employing ethyl chloride as alkylating agent have been performed in dimethyl formamide (DMF) containing 0.05 moles per liter of tetrabutylammonium iodide (TBAI) as the supporting electrolyte, on an Hg electrode. Both the solvent and the electrode material are especially impractical on an industrial scale (Zeitschrift fuer Chemie 1988, 28, 372-373 and Pharmazie 1992, 47, 848-851).

The reactions employing ethyl iodide as alkylating agent have been performed with a large excess of the alkylating agent, typically three to five fold over amine, which makes this process particularly unattractive in view of the high price of the iodide.

Generally, the final thermal splitting of the O-alkyl-carbamates in the manufacture of isocyanates would furnish low boiling ethanol as a by-product which makes the whole process difficult to operate under industrial conditions.

Therefore, higher boiling alcohols like n-butanol would be more preferred. This leads to the pre-requisite in the electrochemical process to employ higher boiling and thus longer chained alkylating agents, e.g., n-butyl chloride.

In preliminary experiments (see Comparative Example 1), it has been found, that n-butyl chloride is much less reactive compared to both ethyl chloride and ethyl iodide and the reaction is very sluggish providing for less than 10% of the target O-butyl-N-alkylcarbamate.

Therefore, the aim of the present invention was to develop a process that can also be conducted on an industrial scale with the use of higher alkylating agents.

### SUMMARY OF THE INVENTION

Surprisingly, it has been found that butyl chloride can be activated at room temperature by addition of an iodide source, as e.g., tetrabutyl ammonium iodide (TBAI) or tetraethyl ammonium iodide (TEN). That increases the isolated yield up to 21% (TBAI) or 40% (TEN), respectively. Most surprisingly, it has been found that butyl chloride can be activated for this synthesis by heat treatment. In this case, the addition of an iodide source is not necessary. The post-treatment with heat yields up to >87% of the desired bis-carbamates. When an iodide source, e.g., TBAI or TEAI are used and the synthesis is carried out under heat treatment the highest yields are reached (up to 95%).

### DETAILED DESCRIPTION OF THE INVENTION

Accordingly the present invention provides an electrochemical process for preparing bis-O-alkyl-carbamates from primary diamines with CO₂ as carbonyl source, characterized in that at least one alkyl halide with at least three carbon atoms in the alkyl group is used as alkylating agent in the presence of at least one iodide source and that the process is carried out at 10 to 215° C., preferably 20 to 215° C., more preferably at 6 0 to 215° C., most preferably at 60 to 195° C., utmost particularly preferred at 60 to 150° C.

Suitable iodide sources are, e.g., symmetrically substituted tetraorganyl ammonium iodides, e.g. tetramethyl-, tetraethyl-, tetrapropyl-, tetrabutyl-, and tetraphenyl ammonium iodides, unsymmetrically substituted tetraorganyl ammonium iodides, e.g., triphenyl methyl ammonium iodide, trimethyl benzyl ammonium iodide, triethyl methyl ammonium iodide, or ethyltrimethylammonium iodide. Preferred tetraorganyl ammonium iodides are tetrabutyl ammonium iodide (TBAI) and tetraethyl ammonium iodide (TEAI).

Further suitable iodide sources are, e.g., sodium iodide or potassium iodide. When sodium iodide and/or potassium iodide are used, they are preferably used in pure form or in association with 15-crown-5 or 18-crown-6. Thereby, their solubility can be enhanced.

The iodide source is used in an amount of at least 0.5 mol-%, related to the alkyl halide/ the sum of the alkyl halides. If two or more iodide sources are used the above specified amount of at least 0.5 mol-% refers to the sum of these iodides.

Preferably the following amounts of iodide/es related to the alkyl halide/es are used (in rising preference): at least 0.5 mol-%, 0.5 to 80 mol-%, 0.5 to 50 mol-%, 0.5 to 20 mol-%, 0.5 to 10 mol-%, 0.5 to 5 mol-%, 1 to 5 mol-%.

In an alternative embodiment of the invention at least one alkyl halide with at least three carbon-atoms in the alkyl group is used and the alkyl halide is activated by heat treatment at 60 to 215° C., preferably at 60 to 195° C., more preferably at 60 to 150° C.. In this case no iodide source is added.

Suitable primary diamines for the above described process are aliphatic and aromatic diamines, preferred primary diamines are 1,6-diaminohexane, 4,4′-methylenebis(cyclohexylamine), 5-amino-1,3,3-trimethylcyclohexanemethylamine, 1,4-diaminobenzene, 2,4-diaminotoluene.

The alkyl halides, which are used in the inventive process, have alkyl groups with at least three carbon-atoms, preferably with at least four carbon atoms.

Examples for suitable alkyl halides are alkyl iodides and alkyl chlorides such as, n- or iso-propyl iodide, n- or iso-propyl chloride, n-, iso- or t-butyl iodide and n-, iso- or t-butyl chloride; preferred alkyl halides are alkyl chlorides, particularly preferred is n-, iso- or t-butyl chloride, most preferred is n-butyl chloride.

According to the understanding of the present invention alkyl iodides do not belong to the iodide sources.

The alkyl halides can be used in different amounts. However, it is further preferred that the alkyl halides are used in such amounts that the groups of the alkyl halides are in a 2.5 molar excess, preferably in a 2.0 molar excess, over the amino groups of the primary diamines. Thereby, the efficiency of the whole process can be further improved.

The syntheses are carried out in solvents. Suitable solvents are for example DMF, DMSO, 1,2-dimethoxy ethane or N-methyl-2-pyrrolidone. Another suitable solvent is acetonitrile. Further suitable solvents are ionic liquids, for example 1-butyl-3-methylimidazolium tetrafluoroborate.

In the cases wherein the activation of the alkyl halide is carried out under heat treatment, it is preferred to work under reflux.

Moreover the syntheses can be carried out under normal pressure (1 atm), reduced pressure or increased pressure, preferably under normal pressure (1 atm) or increased pressure. The temperatures given above refer to syntheses which are carried out under normal pressure.

Suitable electrode materials are for example copper, platinum, zinc, nickel, iron, steel, graphite, glassy carbon and lead.

### EXAMPLES

Details for the measurements of analytics: Electrolysis under galvanostatic control were carried out with an AMEL 552 potentiostat equipped with an AMEL 721 integrator; ¹H and ¹³C NMR spectra were recorded on a BRUKER AC 200 spectrometer using CDCl₃ as internal standard.

### Electrochemical Cell

The cell is composed of a beaker, which contains the copper cathode (A≅10 cm²), covered with a three-necked lid. A glass tube, equipped with a glass frit, is filled with methylcellulose gel and contains the platinum anode (apparent area: A≅1 cm²). The gas inlet is provided by a pipette which dips into the solution, while the gas outlet is ensured by a side-necked plug.

### Methylcellulose Gel

Prepared with 1M solution of tetraethylammonium chloride in DMF (7 g methylcellulose/100 mL solution).

### I. General Protocol for the Preparation of Alkyl Carbamate Diesters (without Addition of an Iodide-Source)

### Inventive: Under Heat Treatment—Comparative: No Heat Treatment

In a two-compartment electrochemical cell, 20 to 30 mL of 0.1 M of tetraethyl ammonium tetrafluoroborate in CH₃CN were added to the cathodic compartment and gaseous CO₂ was bubbled in. A 25 mA current was applied (J=25 mA·cm⁻²) until the consumption of 300 C (3.0 Faradays per mole of amino-group). The current was stopped, the anodic compartment was removed and the solution was flushed with a nitrogen stream for 10 seconds. The diamine (0.5 mmol) was added to the catholyte and the solution was kept under nitrogen atmosphere while stirring. After 1 hour, the cathode was removed and n-butyl chloride (5.0 mmol) was added. In the control experiment (Comparative Example 1) the mixture was stirred overnight at room temperature. In the experiments according to the invention the reaction mixture was refluxed for 3 hours and subsequently stirred overnight at room temperature.

The catholyte was transferred thereafter into a round-bottom flask and the solvent was evaporated under reduced pressure. The remaining solid was extracted with ethyl acetate (3 times) and the organic layers were combined and evaporated in vacuo. The crude reaction was purified by flash column chromatography (silica gel, AcOEt:n-hexane) to afford the pure carbamate diesters.

### Dibutyl hexane-1,6-diyldicarbamate (Comparative Example—Inventive Example)

White powder

### Comparative Example 1: 9% yield

Example 2, according to the invention: 82% yield

¹H-NMR (200 MHz, CDCl₃) δ4.83; (bs, 2H), 4.00; (t, 4H), 3.12; (app. q, 4H), 1.58-1.26; (m, 16H, overlapped with H₂O signal), 0.89; (t, 6H); ¹³C-NMR (200 MHz, CDCl₃) δ156.9, 64.5, 40.7, 31.1, 29.9, 26.3, 19.1, 13.7.

Rf=0.3 (n-hexane: ethyl acetate 7:3).

### Dibutyl (methylene-bis(cyclohexane-4,1,diyl))dicarbamate (Example 3, According to the Invention)

Off-white waxy solid, ≥85% yield

¹H-NMR (200 MHz, CDCl₃) δ4.80; (bd, 1H), 4.55; (bd, 1H), 4.02; (bt, 4H), 3.75; (bs, 1H), 3.39; (bs, 1H), 1.99-1.94; (m, 2H), 1.73-1.00; (m, 26H, overlapped with H₂O signal), 0.91; (t, 6H);

¹³C-NMR (200 MHz, CDCl₃) δ156.0, 64.5, 50.3, 46.9, 44.0, 42.9, 33.7, 33.6, 33.4, 32.7, 32.0, 31.1, 29.7, 28.0, 19.1, 13.8.

Rf=0.3 (n-hexane: ethyl acetate 8:2).

### Butyl((5-((butoxycarbonyl)amino)-1,3,3-trimethylcyclohexyl)methyl)carbamate (Example 4, According to the Invention)

Yellowish oil, ≥87% yield

¹H-NMR (200 MHz, CDCl3) δ4.77; (bt, 1H), 4.50; (bd, 1H), 4.07-4.00; (m, 4H), 3.77; (bs, 1H), 3.26; (d, J=6.2 Hz, 0.4H), 2.9; (d, J=6.6 Hz, 1.5H), 1.74-1.16; (m, 11 H, overlapped with H2O signal), 1.05 (app s, 6H), 0.95-0.88; (m, 12H);

¹³C-NMR (200 MHz, CDCl3) δ157.2, 157.1*, 156.0, 64.7, 64.5, 54.8, 47.5*, 47.1, 46.4, 44.5, 42.7*, 41.9, 36.4, 35.0, 31.9, 31.8*, 31.1, 29.7, 27.6, 23.2, 19.1, 17.7*, 13.8, 12.3*.

Rf=0.3 and 0.2—pair of diastereomers—(n-hexane: ethyl acetate 8:2)

*minor diastereomers

### Dibutyl (1,3-phenylenebis(methylene))dicarbamate. (Example 5, According to the Invention)

White solid, 82% yield

¹H NMR (200 MHz, CDCl3) δ7.22-7.11; (m, 4H), 5.41; (bs, 2H), 4.24; (d, J=5.8 Hz, 4H), 4.01; (t, J=6.5 Hz, 4H), 1.58-1.47; (m, 4H), 137-1.25; (m, 4H), 0.88; (t, J=7.2 Hz, 6H); ¹³C NMR (200 MHz, CDCl3) δ156.9, 139.2, 128.8, 126.4, 64.8, 44.8, 31.0, 19.0, 13.7. Rf=0.1 (n-hexane: ethyl acetate 8:2).

### Synthesis of dibutyl hexane-1,6-diyldicarbamate with Butyl Chloride in DMF

In a two-compartment electrochemical cell (copper cathode and platinum anode) 25 mL of 0.1 M of tetraethylammonium tetrafluoroborate in DMF were added to the cathodic compartment and gaseous CO₂ was bubbled in. A 25 mA current was applied (J=25 mA·cm⁻²) until the consumption of 300 C (3.0 Faradays per mole of amino-group). The current was stopped, the anodic compartment and the copper cathode were removed and the solution was flushed with a nitrogen stream for 10 minutes. The diamine (0.5 mmol hexamethylenediamine) was added to the catholyte and the solution was kept under nitrogen atmosphere while stirring. After 1 hour, n-butyl chloride (5.0 mmol) was added and the reaction mixture was kept for 3 hours at 130° C. and subsequently stirred overnight at room temperature. The catholyte was transferred thereafter into a round-bottom flask and the solvent was evaporated under reduced pressure. The remaining solid was extracted with ethyl acetate (3 times) and the organic layers were combined and evaporated in vacuo. The crude reaction was purified by flash column chromatography (silica gel, AcOEt:n-hexane) to afford the pure carbamate diester in 95% yield.

### II. Preparation of Alkyl Carbamate Diesters (Under Use of Ammonium Iodide)

### Synthesis of Butyl Dicarbamates with Butyl Chloride (TBAI, Catalytic Amount)—Room Temperature (Example 6—Inventive)

In a two-compartment electrochemical cell, 20 to 30 mL of 0.1 M of tetraethylammonium tetrafluoroborate in CH₃CN were added to the cathodic compartment and gaseous CO₂ was bubbled in. A 25 mA current was applied (J=25 mA·cm⁻2) until the consumption of 300 C (3.0 Faradays per mole of amino-group). The current was stopped, the anodic compartment was removed and the solution was flushed with a nitrogen stream for 10 seconds. Hexamethylenediamine (0.5 mmol) was added to the catholyte and the solution was kept under nitrogen atmosphere while stirring. After 1 hour, the cathode was removed and n-butyl chloride (5.0 mmol) was added along with tetrabutylammonium iodide (1-5 mol %). The solution was allowed to stand overnight at room temperature under constant stirring. The catholyte was transferred into a round-bottom flask and the solvent was evaporated under reduced pressure. The remaining solid was extracted with ethyl acetate (3 times) and the organic layers were combined and evaporated in vacuo. The crude reaction was purified by flash column chromatography (silica gel, hexane:AcOEt 8:2) to afford the pure carbamate diester as a white powder in 21% yield.

### Synthesis of Butyl Dicarbamates with Butyl Chloride (TBAI, Catalytic Amount)—80° C. (Example 7—Inventive).

In a two-compartment electrochemical cell typically 20 to 30 mL of 0.1 M of tetraethylammonium tetrafluoroborate in CH₃CN were added to the cathodic compartment and gaseous CO₂ was bubbled in. A 25 mA current was applied (J=25 mA·cm⁻²) until the consumption of 300 C (3.0 Faradays per mole of amino-group). The current was stopped, the anodic compartment was removed and the solution was flushed with a nitrogen stream for 10 seconds. Hexamethylenediamine (0.5 mmol) was added to the catholyte and the solution was kept under nitrogen atmosphere while stirring. After 1 hour, the cathode was removed and n-butyl chloride (5.0 mmol) was added along with tetrabutylammonium iodide (1-5 mol %). The solution was heated to 80° C., then the catholyte was transferred into a round-bottom flask and the solvent was evaporated under reduced pressure. The remaining solid was extracted with ethyl acetate (3 times) and the organic layers were combined and evaporated in vacuo. The crude reaction was purified by flash column chromatography (silica gel, hexane:AcOEt 8:2) to afford the pure carbamate diester as a white powder in >90% yield.

### Synthesis of Butyl Dicarbamates with Butyl Chloride (TEAI as Supporting Electrolyte)—Room Temperature (Example 8—Inventive)

In a two-compartment electrochemical cell typically 20 to 30 mL of 0.1 M of tetraethylammonium iodide in CH₃CN were added to the cathodic compartment and gaseous CO₂ was bubbled in. A 25 mA current was applied (J=25 mA·cm⁻²) until the consumption of 300 C (3.0 Faradays per mole of amino-group). The current was stopped, the anodic compartment was removed and the solution was flushed with a nitrogen stream for 10 seconds. Hexamethylenediamine (0.5 mmol) was added to the catholyte and the solution was kept under nitrogen atmosphere while stirring. After 1 hour the cathode was removed, n-butyl chloride (5.0 mmol) was added and the solution was allowed to stand overnight at room temperature under constant stirring. The catholyte was transferred into a round-bottom flask and the solvent was evaporated under reduced pressure. The remaining solid was extracted with ethyl acetate (3 times) and the organic layers were combined and evaporated in vacuo. The crude reaction was purified by flash column chromatography (silica gel, hexane:AcOEt 8:2) to afford the pure carbamate diester as a white powder in about 40% yield.

### Synthesis of Butyl Dicarbamates with Butyl Chloride (TEAI as Supporting Electrolyte)—80° C. (Example 9—Inventive).

In a two-compartment electrochemical cell typically 20 to 30 mL of 0.1 M of tetraethylammonium iodide in CH₃CN were added to the cathodic compartment and gaseous CO₂ was bubbled in. A 25 mA current was applied (J=25 mA·cm⁻²) until the consumption of 300 C (3.0 Faradays per mole of amino-group). The current was stopped, the anodic compartment was removed and the solution was flushed with a nitrogen stream for 10 minutes. Hexamethylenediamine (0.5 mmol) was added to the catholyte and the solution was kept under nitrogen atmosphere while stirring. After 1 hour the cathode was removed, n-butyl chloride (5.0 mmol) was added and the solution was heated at 80° C. for 2 hours. The catholyte was transferred into a round-bottom flask and the solvent was evaporated under reduced pressure. The remaining solid was extracted with ethyl acetate (3 times) and the organic layers were combined and evaporated in vacuo. The crude reaction was purified by flash column chromatography (silica gel, hexane:AcOEt 8:2) to afford the pure carbamate diester as a white powder in >95% yield.

Dibutyl hexane-1,6-diyldicarbamate.¹H NMR (200 MHz, CDCl3) 54.83; (bs, 2H), 4.00; (t, 4H), 3.12; (app. q, 4H), 1.58-1.26; (m, 16H, overlapped with H2O signal), 0.89; (t, 6H); ¹³C NMR (200 MHz, CDCl3) 5156.9, 64.5, 40.7, 31.1, 29.9, 26.3, 19.1, 13.7. Rf=0.3 (n-hexane: ethyl acetate 7:3).

### Synthesis of Dibutyl Hexane-1,6-diyldicarbamate with Butyl Chloride (TBAI, Catalytic Amount) in DMF

In a two-compartment electrochemical cell (copper cathode and platinum anode) 25 mL of 0.1 M of tetraethylammonium tetrafluoroborate in DMF were added to the cathodic compartment and gaseous CO2 was bubbled in. A 25 mA current was applied (J=25 mA·cm−²) until the consumption of 300 C (3.0 Faradays per mole of amino-group). The current was stopped, the anodic compartment and the copper cathode were removed and the solution was flushed with a nitrogen stream for 10 minutes. The diamine (0.5 mmol hexamethylenediamine) was added to the catholyte and the solution was kept under nitrogen atmosphere while stirring. After 1 hour, n-butyl chloride (5.0 mmol) was added along with tetrabutylammonium iodide (3 mol %) and the reaction mixture was kept for 3 hours at 130° C. and subsequently stirred overnight at room temperature. The catholyte was transferred thereafter into a round-bottom flask and the solvent was evaporated under reduced pressure. The remaining solid was extracted with ethyl acetate (3 times) and the organic layers were combined and evaporated in vacuo. The crude reaction was purified by flash column chromatography (silica gel, AcOEt:n-hexane) to afford the pure carbamate diester in 93% yield.

### III. Example for Synthesis in Cell Without the Gel

### Pseudo-Undivided Cell Configuration

Electrochemical cell. The cell is an H-type glass tube endowed with a glass frit (porosity: 2). Each of the two sides (internal diameter=1.4 cm) contains one electrode: a copper bar—as the cathode—and a glassy carbon bar—as the anode—(A=3 cm², depending on the amount of solvent).

The gas inlet is provided by glass pipettes on both sides of the cell.

### General Protocol to Alkyl Carbamate Diesters in a Pseudo-Undivided Cell

In an H-type electrochemical cell (as described above), 10 mL of 0.1 M of tetraethylammonium chloride in CH₃CN were added to both the sides. Gaseous CO₂ was bubbled to the cathodic side, while nitrogen to the anodic one. A 50 mA current was applied J=15 mA·cm⁻² until the consumption of 300 C (3.0 Faradays per mole of amino-group).* The current was stopped, the electrodes removed and the solution in the anodic side was replaced with fresh 0.1 M of tetraethylammonium chloride in CH₃CN. The catholyte was flushed with a nitrogen stream for 10 minutes before adding the diamine (0.5 mmol) and then the solution was kept under nitrogen atmosphere while stirring. After 1 hour, the alkylating agent (5.0 mmol) was added. When butyl iodide was used, the solution was allowed to stand overnight at room temperature under constant stirring while, in the case of n-butyl chloride, the reaction mixture was refluxed for 3 hours and subsequently stirred overnight at room temperature. The catholyte was transferred into a round-bottom flask and the solvent was evaporated under reduced pressure. The remaining solid was extracted with ethyl acetate (3 times) and the organic layers were combined and evaporated in vacuo. The crude reaction was purified by flash column chromatography (silica gel, AcOEt:n-hexane) to afford the pure carbamate diesters. *during the electrolysis, supplement of tetraethylammonium chloride in the anodic side was required to avoid solvent migration towards the cathode.

This specification has been written with reference to various non-limiting and non-exhaustive embodiments. However, it will be recognized by persons having ordinary skill in the art that various substitutions, modifications, or combinations of any of the disclosed embodiments (or portions thereof) may be made within the scope of this specification. Thus, it is contemplated and understood that this specification supports additional embodiments not expressly set forth herein. Such embodiments may be obtained, for example, by combining, modifying, or reorganizing any of the disclosed steps, components, elements, features, aspects, characteristics, limitations, and the like, of the various non-limiting embodiments described in this specification. In this manner, Applicant(s) reserve the right to amend the claims during prosecution to add features as variously described in this specification, and such amendments comply with the requirements of 35 U.S.C. § 112(a), and 35 U.S.C. § 132(a).

Various aspects of the subject matter described herein are set out in the following numbered clauses:

Clause 1. Electrochemical process for preparing bis-O-alkyl-carbamates from primary diamines with CO2 as carbonyl source, characterized in that at least one alkyl halide with at least three carbon-atoms in the alkyl group is used as alkylating agent in the presence of at least one iodide source and that the process is carried out at 10 to 215° C.

Claus 2. Electrochemical process according to clause 1, wherein the process is carried out at 20 to 215° C., preferably at 60 to 215° C., more preferably at 60 to 195° C. and most preferably at 60 to 150° C.

Clause 3. Electrochemical process according to one of clauses 1 or 2, wherein the iodide source/es is/are used in an amount of at least 0.5 mol-%, more preferably of 0.5 to 5 mol-%, most preferably of 1 to 5 mol-% related to the alkyl halide/ the sum of the alkyl halides.

Clause 4. Electrochemical process according to any of one of clauses 1 to 3, wherein the iodide source/es is/are symmetrically-substituted tetralkyl ammonium iodides, asymmetrically-substituted tetralkyl ammonium iodides and/or sodium iodide, preferably in pure form or in association with 15-crown-5 or 18-crown-6, and/or potassium iodide, preferably in pure form or in association with 15-crown-5 or 18-crown-6.

Clause 5. Electrochemical process according to any of one of clauses 1 to 3, wherein the iodide source(s) is/are tetrabutyl ammonium iodide (TBAI) and/or tetraethyl ammonium iodide (TEN).

Clause 6. Electrochemical process for preparing bis-O-alkyl-carbamates from primary diamines with CO₂ as carbonyl source, characterized in that at least one alkyl halide with at least three carbon-atoms in the alkyl group is used and the alkyl halide is activated by heat treatment at 60 to 215° C., preferably at 60 to 195° C., more preferably at 60 to 150° C.

Clause 7. Process according to any of one of clauses 1 to 6, wherein aliphatic and aromatic primary diamines are used.

Clause 8. Process according to any of one of clauses 1 to 6, wherein 1,6-diaminohexane, 4,4′-methylenebis(cyclohexylamine), 5-Amino-1,3,3-trimethylcyclohexanemethyl-amine, 1,4-diaminobenzene and/or 2,4-diaminotoluene are used as primary diamine/s.

Clause 9. Process according to any of one of clauses 1 to 8, wherein alkyl iodides and/or alkyl chlorides, preferably alkyl chlorides, are used as alkyl halides.

Clause 10. Process according to any of one of clauses 1 to 8, wherein n-, iso- and/or t-butyl chloride, preferably n-butyl chloride, is used as alkyl halide.

Clause 11. Process according to any of one of clauses 1 to 10, wherein the synthesis is carried out in a solvent.

Clause 12. Process according to clause 11, wherein DMF, DMSO, 1,2-dimethoxy ethane or N-methyl-2-pyrrolidone is used as solvent.

Clause 13. Process according to clause 11, wherein acetonitrile is used as solvent.

Clause 14. Process according to any of one of clauses 11 to 13, wherein the heat treatment is carried under reflux.

Clause 15. Process according to any of one of clauses 11 to 14, wherein the synthesis is carried out under normal pressure (1 atm) or increased pressure.

1. An electrochemical process for preparing bis-O-alkyl-carbamates comprising alkylating a primary diamines with CO₂ as carbonyl source and with an alkylating agent containing at least one alkyl halide having at least three carbon atoms in the alkyl group in the presence of at least one iodide source, wherein the process is carried out at 10° C. to 215° C.

2. The electrochemical process according to claim 1, wherein the process is carried out at 20° C. to 215° C.

3. The electrochemical process according to claim 1, wherein the iodide source is used in an amount of at least 0.5 mol-%, relative to the alkyl halide/the sum of the alkyl halides.

4. The electrochemical process according to claim 1, wherein the iodide source is selected from the group consisting of symmetrically-substituted tetralkyl ammonium iodides, asymmetrically-substituted tetralkyl ammonium iodides and sodium iodide.

5. The electrochemical process according to claim 1, wherein the iodide source is selected from the group consisting of tetrabutyl ammonium iodide (TBAI) and tetraethyl ammonium iodide (TEAI).

6. The electrochemical process according to claim 1, wherein the alkyl halide is activated by heat treatment at 60° C. to 215° C.

7. The electrochemical process according to claim 1, wherein the primary diamine is selected from aliphatic primary diamines and aromatic primary diamines.

8. The electrochemical process according to claim 1, wherein the primary diamine is selected from the group consisting of 1,6-diaminohexane, 4,4′-methylenebis(cyclohexylamine), 5-Amino-1,3,3-trimethylcyclo-hexanemethyl-amine, 1,4-diaminobenzene and 2,4-diaminotoluene.

9. The process according to claim 1, wherein the alkyl halide is selected from the group consisting of alkyl iodides and alkyl chlorides.

10. The process according to claim 1, wherein the alkyl halide is selected from the group consisting of n-butyl chloride, iso-butyl chloride and t-butyl chloride.

11. The process according to claim 1, wherein the process is carried out in a solvent.

12. The process according to claim 11, wherein the solvent is selected from the group consisting of DMF, DMSO, 1,2-dimethoxy ethane and N-methyl-2-pyrrolidone.

13. The process according to claim 11, wherein the solvent is acetonitrile.

14. The process according to claim 6, wherein the heat treatment is carried under reflux.

15. The process according to claim 1, wherein the process is carried out under normal pressure (1 atm).

16. The electrochemical process according to claim 1, wherein the process is carried out 60° C. to 195° C.

17. The electrochemical process according to claim 1, wherein the iodide source is in an amount of 1 to 5 mol-% relative to the alkyl halide/the sum of the alkyl halides.

18. The electrochemical process according to claim 4, wherein the iodide source is in pure form.

19. The electrochemical process according to claim 4, wherein the iodide source is in association with one selected from the group consisting of 15-crown-5, 18-crown-6, and potassium iodide.
